(19) United States
(12) Patent Application Publication
(10) Pub. No.: US 2017/0112784 A1
(43) Pub. Date: Apr. 27, 2017

(54) Method and Medicament for Pain Management

(71) Applicant: Colin Dunlop, East Ryde (AU)

(72) Inventor: Colin Dunlop, East Ryde (AU)

(21) Appl. No.: 15/400,261

(22) Filed: Jan. 6, 2017

Related U.S. Application Data
(63) Continuation of application No. 12/739,803, filed on Mar. 31, 2011, now abandoned.

(30) Foreign Application Priority Data
Oct. 24, 2007 (AU) 2007905849

(51) Int. Cl. A61K 31/08; A61K 9/00

(52) U.S. Cl. CPC A61K 31/08; A61K 9/007

(57) ABSTRACT

The present invention relates to a method and an apparatus for treating pain in a patient, such as a veterinary patient. The method comprises the step of administering an inhalation anaesthetic at sub-anaesthetic levels to the patient during anaesthesia of the patient. A suitable agent may be methoxyflurane.

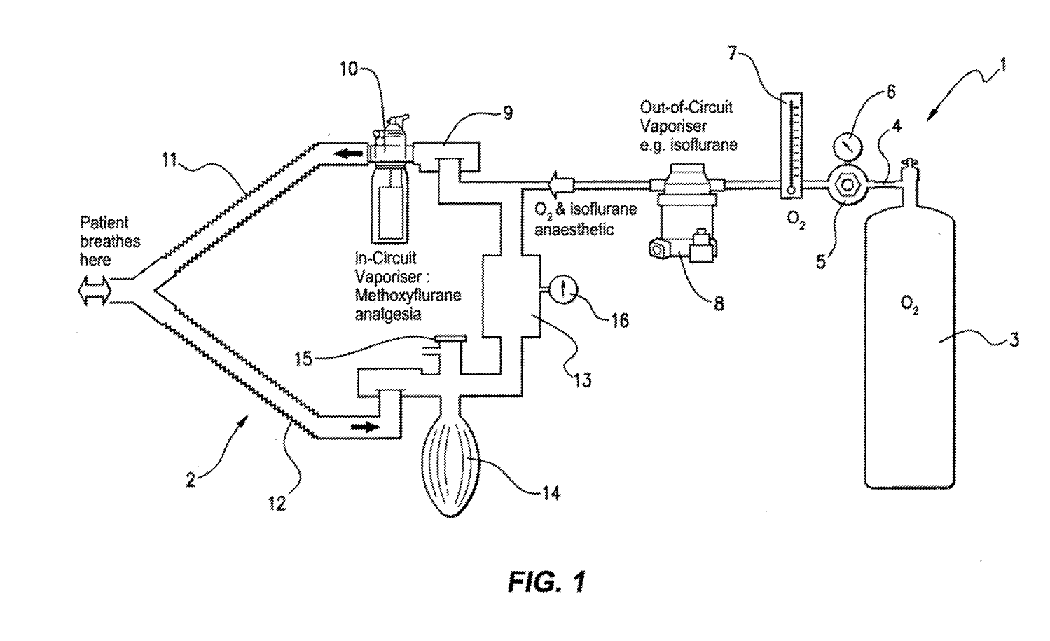

### CROSS-REFERENCE TO RELATED APPLICATIONS

The present application is a continuation of U.S. patent application No. 12/739,803, filed Mar. 31, 2011, which is the U.S. national stage application of PCT Application No. PCT/AU2008/001567, filed Oct. 24, 2008, which claims priority to Australian Patent Application No. 2007905849, filed Oct. 24, 2007, the contents of which are incorporated herein by reference in their entirety.

### FIELD OF THE INVENTION

The present invention relates to a method and medicament for the treatment of pain and, particularly, but not exclusively, to a method and medicament for treating pain following a medical operation on a patient, such as surgery.

### BACKGROUND OF THE INVENTION

Management of pain in patients is very important, particularly during and after treatment of a patient by methods such as surgery.

In animals, for example, pain can cause physical discomfort and stress resulting in high circulating catecholamines, increased vasomotor tone, hyperventilation and hypoglycemia. Pain in animals can slow recovery from an operation and even cause more immediate problems post surgery when an animal may be “waking up” from surgery and experiencing pain. This may result in movements that may lead to complications and an extended period of recovery. Pain alters behaviour including the quality of sleep, feeding and watering intake. All of these factors affect recovery from tissue injury or trauma.

Pain is no less of an issue in humans who may be recovering from trauma for tissue injury or following an operation such as surgery.

Improved management of pain in veterinary and human applications may lead to improved quality of recovery from trauma, such as surgery.

In general veterinary and/or human patient practice analgesic drugs are routinely given to surgical patients in the peri-operative period. In animals, the most commonly used analgesics includes systemic opioids, NSAID's and local or regional nerve blockers. NSAID's such as Carprofen™ or Metacam™ are routinely administered before or during surgery and may be also for 24 to 36 hours post surgery. For major surgery, a variety of techniques are used including opioid, lignocaine and ketamine infusions, local or regional blockers and fentanyl patches. Although these analgesic drugs are useful, it is believed that there exists room for improvement in pain management in veterinary and human treatments, and also room for providing alternative analgesic treatments in order to provide more options for treatment.

In recent years, in both animal and human medicine, there has been general movement away from longer acting anaesthetic agents. There are a number of reasons for this, including occupational health considerations. Long acting anaesthetic agents such as methoxyflurane and many other inhalation anaesthetics have fallen out of favour for a variety of reasons, including environmental toxicity. In laboratory animal anaesthesia, many university animal ethics committees actively discourage the use of anaesthetics with high metabolism or potential occupational health considerations, including methoxyflurane. Methoxyflurane has also been known to cause renal problems in animals and humans.

Fast acting anaesthetics, such as isoflurane or sevoflurane, can lead to problems during recovery. For example, when a horse recovers from anaesthesia, either because of pain or the “shock” of waking, the horse will often try to stand, or move quite quickly after recovery and this may lead to injury. Horses are typically placed in recovery rooms with padded walls in order to minimise trauma on fast recovery from an anaesthetic. Other animals and humans may also experience problems during fast recovery from anaesthesia.

Physical discomfort and stress also occur during euthanasia of animals, particularly where numbers of animals are euthanized simultaneously using noxious inhaled gases such as CO₂ or carbon-monoxide. Pain and stress to animals caused by such a procedure is unnecessarily cruel but also prolongs the time taken to cause death because stress increases cerebral stimulation and circulating catecholamines which cause redistribution of blood flow away from the vital organs where the noxious agent causes its effect in a concentration dependent manner. Prolonged and agonal euthanasia is also undesirable from an occupational perspective because it adds additional pressure to workers already performing a high-stress job.

### SUMMARY OF THE INVENTION

In accordance with a first aspect, the present invention provides a method of treating pain in a patient, comprising the step of administering an inhalation anaesthetic at sub-anaesthetic levels to the patient during anaesthesia of the patient.

In embodiments, suitable inhalation anaesthetics include methoxyflurane, diethyl ether, trichloroethylene, chloroform,and others.

The applicants have found that administering, for example, methoxyflurane at sub-anaesthetic levels can produce affects on patients post-anaesthesia in relation to pain management. It is also thought that it may have an analgesic effect during anaesthesia. Pain may therefore be reduced or prevented during and post-anaesthesia. An additional advantage is that because the methoxyflurane (or other inhalation anaesthetic) is used at sub-anaesthetic levels, the health effects associated with methoxyflurane in use as an anaesthetic are reduced or avoided (eg renal problems, environmental toxicity). A further advantage of at least an embodiment is that methoxyflurane or other long acting inhalation anaesthetics like methoxyflurane, because they are long acting, slow recovery from anaesthesia. Patients are likely to remain in a calm state for longer, therefore, because their pain is being managed and their recovery from anaesthesia is slow, reducing the chances of the patient causing themselves harm because of movements brought on by rapid, painful recovery from anaesthesia.

The inhalation anaesthetic may be administered during the entire period of anaesthesia or only part or parts of the period. In an embodiment, methoxyflurane or other inhalation anaesthetic is administered towards or at the end of the anaesthesia. In an embodiment methoxyflurane is administered at any time during the peri-operative period. In some embodiments, methoxyflurane is administered before, during and/or after anaesthesia.

In an embodiment, the method comprises the further step of administering the inhalation anaesthetic post-anaesthesia to maintain an analgesic effect. In an embodiment the inhalation anaesthetic administered post-anaesthesia is administered at sub-anaesthetic dosages.

In an embodiment, where the inhalation anaesthetic is methoxyflurane, the dosages of methoxyflurane administered may be between 0.01 and 0.7 MAC (minimum alveoli concentration). In an embodiment, the dosage is between 0.05 and 0.65 MAC. In an embodiment, the dosage is between 0.1 and 0.6 MAC. In an embodiment, the dosage is between 0.2 and 0.5 MAC.

The applicants believe that methoxyflurane and other long acting anaesthetics will exhibit an analgesic effect in patients for a relatively long period of time following anaesthesia, when administered at sub-anaesthetic levels.

During anaesthesia, the inhalation anaesthetic, such as methoxyflurane, may be used together with any anaesthetic. In an embodiment, the anaesthetic may be isoflurane, halothane, sevoflurane, desflurane, or any other anaesthetic (including all fast acting anaesthetics).

In one embodiment, the anaesthetic may be delivered at sub-anaesthetic levels to reduce pain during a euthanasia process, for example, for animals such as lab animals. The sub-anaesthetic doses may be delivered together with a noxious agent such as CO₂ or carbon monoxide or other noxious agent. Advantageously, pain and stress is reduced prior to enduring euthanasia.

The patient may be an animal patient and the method may be applied in veterinary medicine. Alternatively, the patient may be a human patient, in human medicine.

In accordance with a second aspect, the present invention provides use of an inhalation anaesthetic in the manufacture of a medicament for the treatment of pain, wherein the medicament is administered at sub-anaesthetic levels in combination with an anaesthetic for anaesthetising the patient.

The medicament may be administered during anaesthesia of a patient, throughout the anaesthesia or at any time during anaesthesia, for example, towards the end of anaesthesia.

In an embodiment, the medicament is administered post-anaesthesia to maintain an analgesic effect.

The patient may be an animal patient or a human patient.

In an embodiment, the inhalation anaesthetic is methoxyflurane.

In accordance with a third aspect, the present invention provides use of an inhalation anaesthetic in the manufacture of a medicament for the treatment of pain following surgery, wherein the medicament is administered at a sub-anaesthetic dosage in combination with an anaesthetic for anaesthetising the patient, during the surgery.

The medicament may be administered throughout the surgery, or at any time during the surgery, for example towards the end of the surgery.

In an embodiment, the dosage is between 0.01 to 0.7 MAC. In an embodiment between 0.05 to 0.65 MAC. In an embodiment between 0.1 and 0.6 MAC. In an embodiment between 0.2 and 0.5 MAC.

In an embodiment, the inhalation anaesthetic is methoxyflurane.

In accordance with a fourth aspect, the present invention provides inhalation anaesthetic for use in the treatment of pain, wherein the inhalation anaesthetic is administered at a sub-anaesthetic dosage in combination with an anaesthetic for anaesthetising the patient.

The inhalation anaesthetic may be administered during anaesthesia in a patient. It may be administered throughout the anaesthesia. Alternatively it may administered at any time during the anaesthesia. In an embodiment, it is administered towards the end of the anaesthesia.

In an embodiment, the inhalation anaesthetic is also administered post-anaesthesia to maintain an analgesic effect.

In an embodiment, the dosage of the inhalation anaesthetic is between 0.01 to 0.7 MAC. In an embodiment between 0.05 to 0.65 MAC. In an embodiment between 0.1 and 0.6 MAC. In an embodiment between 0.2 and 0.5 MAC.

In an embodiment, the inhalation anaesthetic is methoxyflurane.

In accordance with a fifth aspect, the present invention provides inhalation anaesthetic for use in the treatment of pain following surgery, wherein the inhalation anaesthetic is administered at a sub-anaesthetic dosage in combination with an anaesthetic for anaesthetising the patient, during the surgery.

In an embodiment, the inhalation anaesthetic is administered throughout the anaesthesia during surgery. In an embodiment, the inhalation anaesthetic may be administered at any time during the surgery. In an embodiment it may be administered towards the end of the surgery.

In an embodiment, the inhalation anaesthetic is also administered post-anaesthesia to maintain an analgesic effect.

In an embodiment, the dosage of inhalation anaesthetic administered is between between 0.01 to 0.7 MAC. In an embodiment between 0.05 to 0.65 MAC. In an embodiment between 0.1 and 0.6 MAC. In an embodiment between 0.2 and 0.5 MAC.

In an embodiment, the inhalation anaesthetic is methoxyflurane.

The patient may be a human or animal patient.

In accordance with a sixth aspect, the present invention provides a method of treating pain in veterinary applications, comprising the step of administering an inhalation anaesthetic at sub-anaesthetic levels to veterinary patients.

Inhalation anaesthetics such as methoxyflurane have the advantage of reducing or removing pain when administered at sub-anaesthetic levels. This facilitates pain management in veterinary patients that may have undergone a trauma, for example an accident or surgery.

The inhalation anaesthetic may be administered during surgery. It may be administered at the end of surgery. In an embodiment, the inhalation anaesthetic may be administered after the surgery.

In an embodiment, inhalation anaesthetic may be administered at any time to the animal patient in order to reduce or remove pain.

In an embodiment, the inhalation anaesthetic is administered before or during euthanasia. It may be administered along with a noxious gas or substance to deliver euthanasia. The use of the inhalation anaesthetic advantageously decreases pain and stress before and during euthanasia.

In accordance with a seventh aspect, the present invention provides an apparatus for delivering sub-anaesthetic doses of inhalation anaesthetic for use in the treatment of pain during surgery, the apparatus comprising a vaporiser for delivering the inhalation anaesthetic at sub-anaesthetic doses, and a vaporiser for delivering anaesthetic whereby the anaesthetic and inhalation anaesthetic are delivered in combination to the patient.

In an embodiment, the vaporiser for delivering the inhalation anaesthetic is in-circuit.

In an embodiment, the vaporiser for delivering the anaesthetic for anaesthesia is an out of circuit vaporiser. In an embodiment, it is a precision vaporiser.

In an embodiment, the anaesthetic circuit is a closed circuit.

In a eighth aspect, the present invention provides a method of controlling recovery from an anaesthetic in a patient, comprising the step of administering an inhalation anaesthetic at sub-anaesthetic levels to the patient during anaesthesia of the patient.

In a ninth aspect, the present invention provides use of an inhalation anaesthetic in the manufacture of a medicament for controlling recovery from anaesthesia, wherein the medicament is administered at sub-anaesthetic levels in combination with an anaesthetic for anaesthetising the patient.

In a tenth aspect, the present invention provides inhalation anaesthetic for use in controlling recovery from anaesthesia, wherein the inhalation anaesthetic is administered at a sub-anaesthetic dosage in combination with an anaesthetic for anaesthetising the patient.

In an eleventh aspect, the present invention provides a method of treating pain before or during euthanasia in veterinary applications, comprising the step of administering an inhalation anaesthetic before or during euthanasia, together with a toxic substance for euthanasia, to veterinary patients.

In an embodiment, the inhalation anaesthetic is methoxyflurane.

### BRIEF DESCRIPTION OF THE DRAWINGS

Features and advantages of the present invention will become apparent from the following description of embodiments therefore, by way of example only, with reference to the accompanying drawing, in which the single FIGURE is a diagram of an apparatus in accordance with an embodiment of the present invention for delivering an inhalation anaesthetic at a sub-anaesthetic dosage, in combination with an anaesthetic.

### DETAILS DESCRIPTION OF EMBODIMENTS

Embodiments of the present invention relate to the management of pain and/or management of recovery from anaesthesia by the delivery of sub-anaesthetic levels of inhalation anaesthetic to a patient. The sub-anaesthetic levels of anaesthetic are delivered during anaesthesia, along with delivery of an anaesthetic to cause the anaesthesia. The inhalation anaesthetic is used in these embodiments as an “adjunct to anaesthesia” to cause an analgesic effect in the patient. The use of inhalation anaesthetics as an adjunct to anaesthesia may also slow recovery from the anaesthetic, advantageously leading to a gentler controlled recovery and a lower risk of the patient harming themselves because of a rapid recovery from anaesthesia.

The term “sub-anaesthetic level” means a concentration or dosage of inhalation anaesthetic which is not sufficient to cause a state of anaesthesia. This level will vary from patient to patient, but typically occurs at about 0.75 MAC. The surgical level of anaesthesia is typically 1.2 to 1.3 MAC.

In the following described embodiment, the inhalation anaesthetic used to deliver the analgesic effect/slow recovery from anaesthesia, is methoxyflurane. The present invention is not limited to the use of methoxyflurane. Other inhalation anaesthetics may be used at sub-anaesthetic levels to implement pain management/manage recovery from anaesthesia. Suitable alternative substances to methoxyflurane may include diethyl ether, chloroform, trichlorothylene and others. Suitable properties for the inhalation anaesthetic include having high solubility, so that the anaesthetic will be dissolved in the patient's tissues and persist for a relatively long time period, and having analgesic properties.

The methoxyflurane treatment may be used for both animal patients and human patients who have undergone trauma/surgery and who require a procedure which necessitates use of an anaesthetic. The methoxyflurane may be delivered in small amounts throughout the course of the anaesthesia (at any time during the peri-operative period). Because methoxyflurane is highly soluble in tissues, the analgesic effects will persist for a relatively long period of time, in the order of 12 to 74 hours depending on the patient and the dosage.

Methoxyflurane may be administered simultaneously with the anaesthetic or sequentially with the anaesthetic.

Alternatively, the dosage of methoxyflurane may be given at the end of or towards the end of the anaesthesia. Sufficient dosage is given to produce a persistent analgesic effect for a substantial time (12 to 74 hours, for example) post anaesthesia.

As well as delivering the methoxyflurane sub-anaesthetic dose along with an anaesthetic during anaesthesia, following the procedure further sub-anaesthetic levels of methoxyflurane may be delivered to the patient in order to maintain the analgesic effect.

The anaesthetic used with the methoxyflurane to bring about anaesthesia may be any anaesthetic. In this embodiment the anaesthetic is an inhalation anaesthetic such as isoflurane, sevoflurane, halothane or desflurane (relatively fast acting inhalation anaesthetics).

As discussed above, when methoxyflurane was used as a general anaesthetic there were issues with occupational health and safety, and also renal problems as side-effects with large dosages (e.g. 8 or more MAC hours). Low-dose co-administration of methoxyflurane as an adjunct to anaesthesia, in accordance with this embodiment of the invention, reduces these concerns. Further, simple anaesthesia equipment can be utilized, there is little risk of administration of a hypoxic mixture, occupational health considerations are minimized and waste anaesthetic gas may be easily managed. Advantages of methoxyflurane include:

it can be scavenged in charcoal;

methoxyflurane is a potent compound, so small concentrations are effective;

it will provide intra-operative analgesia (as well as post-operative analgesia) so there will be less “patient wake-up” during surgery, which will also lead to no increase in cost of the primary inhalation anaesthetic (i.e. no increase in dosage of primary inhalation anaesthetic will be required to deal with patient wake-up problems);

co-administration of methoxyflurane as an adjunct to anaesthesia with the primary anaesthetic (which in this embodiment is an inhalation anaesthetic such as isoflurane or sevoflurane) decreases the amount of primary inhalation anaesthetic required and therefore saves cost of expensive anaesthetics such as isoflurane or sevoflurane;

duration of post-anaesthesia analgesia will depend on the concentration of methoxyflurane administered and the duration of administration, but is reasonably expected to be between 8 and 24 hours for a 1 to 2 hour procedure;

the use of the methoxyflurane will slow recovery from the anaesthetic procedure, which means that sharp movements and patients damaging themselves by sharp, quick movements is less likely. This is particularly important for animal patients.

The drawing illustrates an apparatus in accordance with an embodiment of the present invention, which is arranged to facilitate administration of inhalation anaesthetic in accordance with an embodiment of the present invention. In this embodiment the inhalation anaesthetic is methoxyflurane.

The illustrated apparatus includes a delivery system for delivering oxygen and anaesthetic (generally designated by reference numeral 1) to a patient (not shown) via a patient re-breathing circuit generally designated by reference numeral 2. A pressurised source of oxygen 3 is provided. Connected via a line 4 to the source 3 is a regulator 5 for regulating oxygen pressure and a pressure gauge 6. An oxygen flow meter 7 is connected in the line 4 from the regulator 5 to an anaesthetic vaporiser 8.

In this example, the vaporiser 8 is arranged for the delivery of the fast acting inhalation anaesthetic isoflurane. It will be appreciated that the present invention is not limited to any particular anaesthetic to be used in combination with the inhalation anaesthetic for analgesia. Isoflurane is one suitable anaesthetic, sevoflurane is another, and there may be many others. It will depend on the procedure, the patient and the patient requirements. The line 4 connects the output of the vaporiser 8 to the patient re-breathing circuit 2, conveying a mixture of oxygen and isoflurane anaesthetic to the patient re-breathing circuit 2.

The re-breathing circuit 2 comprises a line 9 conveying the oxygen and isoflurane mixture, via in-circuit vaporiser 10 to a line 11 to the patient. A mask or endotracheal tube (not shown) will usually be connected to the line 11 in order to deliver the anaesthetic oxygen to the patient. A return line 12 receives waste gases from the patient. These are conveyed via a vessel 13 which contains soda lime for removing carbon dioxide from the waste gases so that the gases can be re-breathed. Non-return valves (not shown) ensure that gas flow direction in the re-breathing circuit 2 is one way. The re-breathing circuit 2 also includes a reservoir or re-breathing bag 14, which provides a variable storage volume to compensate for variations in sizes of breath for each patient. It also allows for positive ventilation. A “pop-off” valve 15 is also provided for relieving pressure in the circuit if necessary. A pressure gauge 16 is provided to indicate the pressure in the re-breathing circuit 2. A flush by-pass system is also provided (not shown in the diagram) in order to by-pass the vaporiser 8 and provide pure oxygen to the circuit 2 on operation of a flush valve.

In this embodiment, the in-circuit vaporiser 10 is arranged to provide a controlled dose of methoxyflurane, at sub-anaesthetic levels, in order to provide an analgesic effect and optionally also slow recovery from the anaesthesia. As discussed above, the dosage will depend upon a number of factors, including the size of the patient, the type of patient (e.g. animal, human, metabolic rate, etc.), the amount of time that the analgesia effect is required post-anaesthesia, the speed of recovery from anaesthesia that is required. In general, the amount of methoxyflurane dosage will be between 0.01 and 0.7 MAC. In this embodiment it will be in the range of between 0.1 and 0.6 MAC.

The above-described apparatus is one embodiment only of an apparatus for delivery of the inhalation anaesthetic for the purposes of analgesia and/or to slow recovery from anaesthetic. Other methods of delivery and other arrangements for delivery may be utilised. For example, dual out-of-circuit precision vaporisers may be utilised, one for methoxyflurane and one for isoflurane or sevoflurane (or other anaesthetic).

Delivery may alternatively be by injection of the analgesic and/or the anaesthetic. Delivery may be by an inhaler and/or a mask. Delivery post anaesthetic, for example, may be via an inhaler. There may be other methods of delivery that may be utilised.

### Example

Administration of methoxyflurane for a short period at the end of equine anaesthesia to modify recovery from isoflurane or sevoflurane.

As in human medicine, complications can and do arise during anaesthesia and in the recovery period. Mortality rates in veterinary anaesthesia today range from 100 to 500 deaths per 100,000 anaesthetics for pet animals (cats and dogs) to 350 to 1,000 deaths per 100,000 anaesthetised horses.

In horses, recovery to consciousness is a major cause of anaesthesia-related morbidity and mortality, accounting for 50% of horse anaesthesia deaths. Because of their size and potential for injury, horses are typically placed in dimly lit, padded rooms to recover from anaesthesia.

At Randwick Equine Centre in Sydney halothane is routinely administered to 0.01 mg/kg acepromazine & methadone 0.05 mg/kg+/−xylazine 0.2 mg/kg all 1M 15 minutes before the end of the procedure where there is concerns about a poor recovery or a painful procedure where the veterinary surgeon is unable to provide a local block to provide analgesia (eg throat sx).

Halothane use is rapidly declining because of a lack of licensed manufacturers. Halothane has been widely used because horses ventilate well during anaesthesia (so the anaesthetic level is stable) and because it is generally associated with a reasonable quality of recovery, albeit slow (30 to 75 minutes). Slow, reasonable quality recoveries are acceptable, although because the horses are directly monitored at this time, usually by a veterinarian, this is expensive from a man-power perspective.

Isoflurane or Sevoflurane are the anaesthetics being used to replace halothane. Both drugs have faster uptake/distribution so recoveries are faster. This causes a short period; usually 15 to 20 minutes from the end of anaesthesia where the horse will suddenly attempt to stand, may fall once or twice, and then be able to stand in a reasonably stable fashion.

Veterinarians are currently working to change to these newer agents and utilise various methods to modify recovery such as that described above at Randwick Equine Centre. Alternatively, infusions of short acting parenteral drugs such as propofol are being administered for 10 to 15 minutes at the end of anaesthesia to keep the horse “asleep” to allow it to “blow off” the Isoflurane or Sevoflurane.

Veterinarians in equine surgical practice desire a simple method for improving the quality of recovery from equine anaesthesia (by prolonging the “sleep time”).

### Example:

Trial of methoxyflurane administered to horses at the end of anaesthesia, attempting to modify recovery:

On 12 different days MOF was administered to 12 horses at the end of between 1 to 2 hours of isoflurane anaesthesia with the intention of improving the quality of recovery from anaesthesia by slowing the recovery time. Horses recovering from isoflurane typically attempt to stand in 10 to 15 minutes, usually fall down and try to get back up 2 or 3 times (sometimes violent, resulting in injuries from skin abrasions to fractured limbs) and are then standing 15 to 20 minutes after anaesthetic administration ceased. In comparison, halothane results in slower recoveries, typically twice as long as isoflurane. Halothane recoveries are less violent and result in less likelihood of injury.

Initially trials were also performed in anattempt to determine the dose and duration of MOF administration that would result in improved quality of recovery. The trials showed that the MOF dose required was much lower and the duration of MOF administration much shorter than anticipated. The initial trials resulted in long recoveries with marked ataxia, in some cases resulting in minor injury to the horse.

The last 8 horses were administered with ever lower doses of MOF for less time with better results (see table below). There was less ataxia and a better quality recovery although the dose was decreased up until the last horse. The lowest dose produced a good result.

| | Anaesth | | | | | | | |
|---|---|---|---|---|---|---|---|---|
| Wt | duration | Methoxy | 1^{st} | | | | Walk | |
| kgs | Mins. | Total ml | Movt | Stern. | Stnd | Atax | out | Comments |
| 472 | 130 ISO | | 23 | 32 | 63 | 78 | 100 | Try stand at 23 |
| | | | | | | | | min & fell |
| 492 | 75 ISO | 540 | 50 | 60 | 75 | 85 | 100 | Stood 69- fell |
| | | | | | | | | down x 2 |
| 470 | 85 ISO | 210 | 5 | 14 | 29 | 35 | 55 | 10x wobble then |
| | | | | | | | | saw horse |
| 338 | 30 HAL | | 27 | 30 | 31 | Fell | 55 | Stood/fell x 3 |
| | | | | | | | | then stall walk |
| 462 | 135 ISO | 350 | 48 | ND | 49 | 59 | 75 | Still 45 mm then |
| | | | | | | | | stood |
| 492 | 90 HAL | | 30 | 50(2) | 65 | 75 | 85 | Poor - flip over |
| | | | | | | | | 15 mm |
| 580 | 16O ISO | 300 | 37 | 60 | 61 | 85 | 90 | Broke out of |
| | | | | | | | | recov - wreck |
| 332 | 135 ISO | 200 | 17 | 25 | 26 | 30 | 41 | Good |

In addition to slowing recovery, methoxyflurane has the further advantage of providing an analgesic effect for a substantial amount of time after recovery from anaesthesia.

In the above embodiments inhalation anaesthetic is used during surgery. The present invention is not limited to be used during surgery, but may be used in any process where anaesthesia is required.

As also discussed above, the present invention is not limited to the use of methoxyflurane as the inhalation anaesthetic. Other suitable inhalation anaesthetics could be used.

In another embodiment, the use of inhalation anaesthetic at sub-anaesthetic doses or even at anaesthetic doses may be used during or prior to euthanasia using a noxious substance such as a noxious inhaled gas such as CO₂ or carbon monoxide. This may reduce pain and stress to animals caused by such a procedure.

Euthanasia of groups of animals together such as laboratory rats and mice, chickens for disease outbreak control and non-domestic cats at animal control centres, usually involves placing the animals in a chamber and administering a toxic and/or a noxious gas such as CO₂ or carbon monoxide. Traditionally, these noxious agents are administered alone or with small amounts of air (e.g. 20% to prevent death by asphyxia, which is very stressful) despite our knowledge that such deaths appear to be associated with discomfort and/or pain to the animals. Induction of anaesthesia with some inhalation anaesthetic agents such as methoxyflurane advantageously may be stress and pain free.

In this embodiment, methoxyflurane or another inhalation anaesthetic is delivered during or prior or both prior and during administration of toxic or noxious gases to animals being euthanized It may be delivered to animals being euthanized in chambers. In an embodiment, the inhalation anaesthetic it administered with air or oxygen at sub to anaesthetic levels to provide algesia, sub-anaesthesia (sedation) or anaesthesia, prior to administration of lethal concentrations of toxic or noxious gases that will cause death. In this embodiment, the anaesthetic administration is of short term, sufficient to reduce stress during the actually euthanasia caused by a separate noxious gas. The anaesthetic drug of itself could be administered at high concentration to cause death but it would be a slower process, not as reliable, cause significant environmental pollution and potentially occupation exposure or potential for abuse.

In another embodiment, there is simultaneous administration of the anaesthetic agent in an air or oxygen mixture with the noxious gas. For this embodiment, a less soluble (faster acting) inhalation anaesthetic is required, such as sevoflurane, isoflurane or possible halothane.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A method of treating pain in a patient, consisting of administering as an adjunct to anaesthesia by a primary anaesthetic, a controlled dose of an inhalation anaesthetic at sub-anaesthetic levels to the patient during anaesthesia of the patient while the patient is unconscious, to provide an analgesic effect persisting post-anaesthesia based on the type of inhalation anaesthetic administered and duration of administration of the inhalation anaesthetic.

2. The method in accordance with claim 1, wherein the step of administering the inhalation anaesthetic is carried out throughout anaesthesia.

3. The method in accordance with claim 1, wherein the step of administering the inhalation anaesthetic is carried out towards the end of anaesthesia.

4. The method in accordance with claim 1, wherein the step of administering the inhalation anaesthetic continues post-anaesthesia to maintain an analgesic effect.

5. The method in accordance with claim 1, wherein the dosage of the inhalation anaesthetic administered is between 0.01 and 0.7 MAC.

6. The method in accordance with claim 5, wherein the dosage is between 0.05 and 0.65 MAC.

7. The method in accordance with claim 6, wherein the dosage is between 0.1 and 0.6 MAC.

8. The method in accordance with claim 7, wherein the dosage is between 0.2 and 0.5 MAC.

9. The method in accordance with claim 1, wherein the patient is an animal.

10. The method in accordance with claim 1, wherein the patient is a human.

11. The method in accordance with claim 1, wherein the inhalation anaesthetic is methoxyflurane.

12. The method in accordance with claim 1, wherein the dose is determined based on amount of time that the analgesic effect is required post-anaesthesia, type of patient, and size of patient.

13. A method of treating pain in a patient, comprising administering a controlled dose of methoxyflurane (MEOF) at a sub-anaesthetic level to a patient during anaesthesia of the patient while the patient is unconscious, to provide an analgesic effect and slow recovery from anaesthesia.

14. The method in accordance with claim 13, further comprising continuing to administer MEOF at a sub-anaesthetic level after cessation of anaesthesia.

15. The method in accordance with claim 13, wherein the dosage of MEOF administered is between 0.01 and 0.7 MAC.

16. The method in accordance with claim 15, wherein the dosage is between 0.05 and 0.65 MAC.

17. The method in accordance with claim 16, wherein the dosage is between 0.1 and 0.6 MAC.

18. The method in accordance with claim 17, wherein the dosage is between 0.2 and 0.5 MAC.

19. The method in accordance with claim 13, wherein MEOF is not administered prior to from about 1 to about 2 hours before anaesthesia is ceased.
